(19) United States
(12) Patent Application Publication
(10) Pub. No.: US 2002/0022826 A1
(43) Pub. Date: Feb. 21, 2002

(54) Burst electrode

(76) Inventors: John R. Reynolds, Gainsville, FL (US); Hiep Ly, Webster, NY (US); Patrick John Kinlen, Fenton, MO (US)

Correspondence Address: THOMPSON COBURN, LLP, ONE FIRSTAR PLAZA, SUITE 3500, ST LOUIS, MO, 63101, US

(21) Appl. No.: 09/929,197

(22) Filed: Aug. 14, 2001

Related U.S. Application Data
(60) Provisional application No. 60/225,084, filed on Aug. 14, 2000.

(51) Int. Cl. A61K 9/22

(52) U.S. Cl. 604/890.1

(57) ABSTRACT

A burst electrode system is provided that comprises an electroactive polymer having thereon either a polyanionic or polycationic dopant, and a biologically active ingredient that is releasable from said electroactive polymer, whereby said burst electrode system exhibits a release profile greater in quantity and faster than a standard Faradaic profile. The biologically active ingredient preferably is a drug. The electroactive polymer preferably is a polypyrrole or a polypyrrole polyelectrolyte complex such as polypyrrole poly(styrene sulfonate), heparin or polyacrylic acid.

### CROSS-REFERENCES TO RELATED APPLICATIONS

This patent application claims the benefit of priority under 35 U.S.C. §119(e) of United States Provisional Patent Application No. 60/225,084, filed Aug. 14, 2000. U.S. Provisional Patent Application No. 60/225,084 is incorporated by reference herein in its entirety.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

This invention relates to drug release systems, which have nonlinear release rates. More particularly, this invention relates to electrodepositing cationic or anionic drugs onto an electroactive polymer and releasing the drugs in a single burst (i.e. a nonlinear response) by application of a current or potential to the electroactive polymer.

### BACKGROUND OF THE INVENTION

Drug delivery systems have been sought with the goal of attaining a higher degree of control over the amounts, and release rates, of bio-active molecules, which can be supplied to a recipient via the drug delivery system. See, K. Park, Ed.; Controlled Drug Delivery, Challenges and Strategies, ACS Press, Washington, D.C., 1997 and T. Okano, Ed.; Biorelated Polymers and Gels: Controlled Release Applications in Biomedical Engineering, Academic Press, San Diego, 1998. This arises because with conventional drug administration the amount of active molecule in a patient's system increases, reaches a plateau, and subsequently decreases. This “peaking” of concentration can lead to unwanted effects (e.g. drug concentration may attain toxic levels or the rapid loss of drug concentration in the bloodstream can lead to a point where it is ineffective). In addition, drugs that may be effective under certain biophysical conditions, or only in particular areas of the body, may be ineffective or degraded in other areas of the body. As each patient and their respective environmental conditions are different, follow-up on drug administration is necessary, and thus, having an improved control over drug administration is extremely useful.

Controlled release drug delivery is a drug delivery technique, which involves targeting one or more factors including time, course, or the location of drug delivery. The main objective in controlled release is to achieve an effective therapeutic administration of the necessary dosage for an extended period of time and to provide the drug only when and where it is necessary. Controlled drug delivery allows targeting of a drug to a specific organ or part of the body, thereby protecting the drug from biochemical systems which might interact in a negative fashion. Thus, the desired therapeutic effect is attained with a higher degree of accuracy and longer duration than multiple doses of the same drug using standard administration methods. Controlled release methods can supply active molecules at a rate equal to, greater than, or less than that of absorption by the system.

There are several forms of controlled release drug delivery systems. One of these, transdermal delivery, uses the patient's skin as a membrane for partially controlling the rate of drug into the blood. Delivery of a bio-active molecule across the membrane requires energy, which can be induced using several methods including ultrasound, chemical modification of drug(s) and electrical current. See, I. Zhang, K. K. Shung D. A. Edwards. Hydrogels with Enhanced Mass Transfer for Transdermal Drug Delivery. J. Pharmaceutical Sciences 85(12)(1996) 1312-1316 and E. R. Cooper, A. F. Kydonieus and B. Berner (Eds). Transdermal Delivery of Drugs. Vo. 2. CRC Press, Boca Raton, Fla. 1987, pp. 57. A variety of commercial systems are now available using transdermal delivery methods, including scopolamine to treat motion sickness, nitroglycerin for angina, estradiol for postmenopausal syndrome, and clonidine as an antihypertensive. Other controlled release drug delivery systems include ocular delivery, implanted transdermal delivery, and oral delivery, which can be achieved via the chemical modification of drugs and the entrapment of drugs in small vesicles. Ionotophoresis, which uses electric field driven transport of drugs across a membrane, has been used to supply cocaine, epinephrine, penicillin, insulin, pilocarpine and many other drugs to the body. See, M. R. Prausnitz, C. S. Eke, C. H. Liu, J. C. Pang, T. Singh, R. Langer, J. C. Weaver. Transdermal Transport Efficiency During Skin Electroporation and lontophoresis. J. Control. Rel. 38 (1996) 205-217; S. B. Ruddy, B. W. Hadzija. The Role of Stratum Corneum in Electrically Facilitated Transdermal Drug Delivery I. Influence of Hydration, Tape-Stripping and Delipidation on the DC Electrical Properties of Skin. J. Control. Rel. 37 (1995) 225-238; J. Hirvonen, F. Hueber, R. H. Guy. Current Profile Regulates Iontophoretic Delivery of Amino Acids Across the Skin. J. Control. Rel. 37 (1995) 239-249; and A. Jadoul, V. Preat. Electrically enhanced transdermal Delivery of Domperidon. Intl. J. of Pharmaceutics, 154(2) (1997) 229-232. In this field, techniques having more chemically specific and time profile control are needed. Further developments are materials limited providing an opportunity for materials scientists to design new drug release systems.

Electroactive and conductive polymers have attracted attention as candidates for delivery of ionic drug species due to their redox properties, which can allow controlled ion transport from the polymer membrane. See, Y. J. Qiu, J. R. Reynolds. Dopant Anion Controlled Ion Transport Behavior of Polypyrrol. Polym. Eng. And Sci. 31 (1991) 417-421; and J. R. Reynolds, M. Pyo, Y. J. Qiu, Cation and Anion Dominated Ion Transport During Electrochemical Switching of PPy Controlled by Polymer Ion Interaction. Synth. Met. 55-57 (1993) 1388-1395. Redox switching of a conductive polymer membrane in an electrolyte solution allows a number of different oxidation states to be accessible. These redox states are stabilized by charge balancing counterions (often called dopant ions), which move in and out of the film during electrochemical switching. Using these processes, a variety of anions, including but not limited to salicylate, Fe(CN)₆⁻³, glutamate, and ATP can be electrochemically bound into the conductive polymer membrane and released during reduction. See, B. Zinger, L. L. Miller. Timed Release of Chemicals from Polypyrrole Films. J. Am. Chem. Soc. 106 (1984) 6861-6863; A. Boyl, E. Genies, M. Fouletier. Electrochemical Behavior of PPy doped with ATP Anions. J. Electroanal. Chem 279 (1990) 179-186; M. Pyo J. R. Reynolds. Electrochemically Stimulated Adenosine 5′-Triophosphate (ATP) Release Through Redox Switching of Cuncting Polypyrrole Films and Bilayers. Chem. Mater. 8(1996) 128-133; and M. Pyo, G. Maeder, R. T. Kenedy, J. R. Reynolds. Controlled Release of Biological Molecules from Conducting Polymer Modified Electrode The Potential Dependent Release of Adenosine 5′-Triphosphate from Poly(pyrrole adenosine 5′-triphosphate) Films. J. Electroanal, Chem 368 (1994) 329-332. On the other hand, when using electrostatically or physically entrapped and bound dopant anions, materials are prepared that can be used to release cations. In this case, cations are loaded during reduction of the conductive polymer: bound anion material. See, M. Hepel. Composite Polypyrrole Films Switchable Between the Anion and Cation Exchanger States. Electrochemica Acta 41 (1996) 63-76; and M. Hepel, F. Mahdavi. Applications of the Electrochemical Quartz Crystal Microbalance for Electrochemically Controlled Binding and Release of Chlopromazine from Conductive Polymer Matrix. Microchemical J. 56(1997) 54-64. In most instances, the multi-ionic high molecular weight species used are polyelectrolytes including poly(styrene sulfonate)(PSS), nation and heparin. See, L. A. Prezyna, Y. J. Qiu, J. R. Reynolds, G. E. Wnek. Interaction of Cationic Polypeptides with Electroactive Polypyrrole/Polystyrene Sulfonate and Poly(N-methylpyrrol)/Poly(styrenesulfonate) Films. Macromolecules 24 (1991) 5283-5287; L. Miller. Electrochemically Controlled Release of Drug Ions from Conducting Polymers. Mol. Cryst. Liq. Cryst. 160 (1988) 297-301; Q. X. Zhou, L. Miller, J. R. Valentine. Electrochemically Controlled Binding and Release of Protonated Dimethyldopamine and Other Cations from Poly(N-methylpyrrol)/polyanion Composite Redox Polymers. J. Electroanal. Chem. 261 (1989) 147-164; K. Naoi, Lein, M., Smyrl, W. H. J. Electroanal. Chem. 272, (1982) 273; and C. K. Baker, Y. J. Qiu, J. R. Reynolds. Electrochemically Induced Charge and Mass Transport in Polypyrrole/poly(styrene sulfonate) molecular composites. J. Phys. Chem. 95 (1991) 4446-4452. It is well known that polypyrrole (PPy)/PSS films are an example of electroactive polymers having cation dominated transport characteristics. By creating materials with combined cation dominant and anion dominant transport characteristics, electrically conductive polymer membranes can be prepared which can supply either anionic or cationic drugs under application of different applied potentials.

Despite the foregoing progress, a need exists for a drug delivery system, which overcomes the aforementioned deficiencies. See, K. Naoi, Lien, M., Smyrl, W. H. J. Electroanal. Chem. 272, (1982) 273 and C. K. Baker, Y. J. Qiu, J. R. Reynolds, Electrochemically Induced Charge and Mass Transport in Polypyrrole/Poly(styrene sulfonate) Molecular Composites. J. Phys. Chem 95 (1991) 446-4452.

### BRIEF SUMMARY OF THE INVENTION

The invention herein comprises a burst electrode system comprising an electroactive polymer having thereon a biologically active moiety releasable from said electroactive polymer, whereby said burst electrode system exhibits a non Faradaic release profile of biologically active ingredient(s).

The burst electrode system of this invention comprises an electroactive polymer, which has a drug releasable therefrom incorporated into the electroactive polymer. The burst electrode system exhibits a drug release profile characterized generally in FIG. 1.

Also provided in this invention is a method of treating a patient using a burst electrode system. This burst electrode system comprises an electroactive polymer loaded with a drug releasable therefrom. This system can be placed in contact with a patient, so that when the system is triggered, a release of the drug from said electroactive polymer makes the drug effectively available to said patient.

Also described herein is a method for preparing a burst electrode system. This process comprises electropolymerizing pyrrole (for example by constant current polymerization) in a suitable polymerizable pyrrole and polystyrene sulfonate composition to form a polymer. This polymer then is loaded with a releasable drug by reduction of said drug (for example by constant potential reduction) with said polymer in a suitable composition to form an initial electrode system. Thereafter, the initial electrode system is removed from said solution to allow equilibration of said polymer outside said solution.

It is an object of this invention to provide a drug delivery system that provides a high degree of control over the concentration of active molecules which can be supplied by such a system.

It is another object of this invention to create a drug delivery system that provides a high degree of control over the rate at which an active molecule can be supplied by such a system.

It is yet another object of this invention to create a drug delivery system wherein controlled release is utilized to provide an effective therapeutic administration of the necessary dosage for an extended period of time and to provide the drug only when necessary.

These and other objects are provided in this invention that is described in more detail hereafter.

### BRIEF DESCRIPTION OF THE DRAWINGS

FIG. 1 illustrates a general drug release profile for a burst electrode system.

FIG. 2 illustrates the amount of dopamine released from PPy/PSS⁻-dop⁺ at 3.0 μA/cm² in phosphate buffer, (a) theoretical assuming faradaic released, (b) experimental.

FIG. 3 illustrates the pulsatile dopamine release from PPy/PSS⁻-dop⁺ using 5s pulse of 3.3 μA/cm² followed by 60 s open circuit in phosphate buffer.

FIG. 4 illustrates the amount of dopamine released from PPy/PSS⁻-dop⁺ as a function of time upon the application of 3.3 μA/cm² for films of varied thickness. (a) 1.1 μm (b) 2.2 μm (c) 2.9 μm (d) 3.6 μm (e) 4.3 μm (f) 5.8 μm (g) 7.2 μm (h) 9.8 μm (i) 19.1 μm.

FIG. 5 illustrates the pulsatile epinephrine released from PPy/PSS⁻-epi⁺ using a 5s pulse of 3.3 μA/cm² followed by 60 s open circuit in phosphate buffer.

FIG. 6 illustrates the amount of epinephrine released from PPy/PSS -epi⁺ as a function of time upon application of 3.3 μA/cm² for films of varied thickness.

FIG. 7 illustrates the amount of metaproterenol released from PPy/PSS⁻-met⁺ at 3.3 μA/cm² in phosphate buffer.

FIG. 8 illustrates the pulsatile metaproterenol released from PPy/PSS⁻-met⁺ using a 5 s pulse of 3.3 μA/cm² followed by 60 s open circuit in phosphate buffer.

FIG. 9 illustrates the amount ATP released from PPy/ATP film at −0.5 V vs Ag/AgCl in 0.1 M NaCl, (a) immediately after synthesis and (b) after 17 h storage under argon.

FIG. 10 illustrates the UV spectra showing (a) 6×10⁻⁵ M standard ATP solution, along with ATP release from PPy/ATP during potential cycling between −1.0 V and 0.0 V at 20 mV/s, (b) pH 5.6 aqueous 0.1 M NaClO₄ and (c) pH 7.4 phosphate buffer.

FIG. 11 illustrates the amount of ATP released from PPy/ATP as a .function of time in 0.1 M NaCl at different release potentials (a) −0.10 V (b) −0.20 V (c) −0.26 V (d) −0.27 V (e) −0.28 V (f) −0.29 V (g) −0.30 V (h) −0.40 V (h) −0.50 V (i) −0.60 V (k) −0.70 V (1) −0.80 V.

FIG. 12 illustrates pulsatile ATP release from PPy/ATP in 0.1 M NaCl using a 5.0 s pulse at −0.25 V followed by +0.5 V for 30 min.

FIG. 13 illustrates the amount of dopamine released from PNMPy/PSS⁻-dop⁺ at 3.3 μA/cm² in phosphate buffer, (a) immediately after synthesis (b) after storage under argon for 10 days.

FIG. 14 illustrates pulsatile dopamine release from PNMPy/PSS⁻-dop⁺ using a 5 s pulse of 3.3 μA/cm² followed by 60 s open circuit in phosphate buffer.

### DETAILED DESCRIPTION OF THE INVENTION

This invention comprises a burst electrode system comprising an electroactive polymer having thereon either a polyanionic or polycationic dopant and a biologically active moiety releasable from said electroactive polymer. The electroactive polymer is preferably a polypyrrole (PPy) or polypyrrole polyelectrolyte complex. Nonlimiting examples of polypyrrole polyelectrolyte complexes include polypyrrole poly(styrene sulfonate) (PPy)/PSS, heparin and polyacrylic acid. One of skill in the art would recognize that other commonly available materials would also have similar properties and could be used in the practice of the instant invention.

The release occurs in a novel non-Faradaic fashion. The “burst release” that occurs for this invention described herein exhibits a release profile greater in quantity and faster in time than a standard (“linear”) Faradaic profile.

When prepared appropriately, conducting and electroactive polymers can serve as electrically-stimulatable membranes for the inclusion and release of both anionic and cationic species. Polymer:ion interactions are controlled by various chemical properties including size, molecular weight, charge, and the nature of bonding interactions (e.g., H-bonding) between different chemical components. Nonlimiting examples of electroactive conducting polymers useful in the practice of the instant invention include polypyrrole, poly(N-methyl pyrrole), substituted polypyrrole, polythiophene, polydioxythiophene, polyaniline and the like. One of ordinary skill in the art would recognize that other polymers with similar properties would also be useful in the practice of the instant invention.

Biologically active ingredient(s) useful herein is preferably a pharmaceutical (compound) selected from the group comprising N-saids, analgesics, antihistamines, antitussives, decongestants, expectorants, steroids, enzymes, proteins, antibiotics, hormones, and mixtures thereof and the like.

Examples of such useful pharmaceutical compounds include but are not limited to nutritional supplements, anti-inflammatory agents (e.g. NSAIDS such as s-ibuprofen, ketoprofen, fenoprofen, indomethacin, meclofentamate, mefenamic acid, naproxen, phenylbutazone, piroxicam, tolmetin, sulindac, and dimethyl sulfoxide), antipyretics, anesthetics including benzocaine, pramoxine, dibucaine, diclonine, lidocaine, mepiracaine, prilocaine, and tetracaine; demulcents; analgesics including opiate analgesics, non-opiate analgesics, non-narcotic analgesics including acetaminophen and astringent including calamine, zinc oxide, tannic acid, Hamamelis water, zinc sulfate; natural or synthetic steroids including triamcinolone, acetonide, perdnisone, beclomethasone dipropionate; asthmatic drugs including terbutaline sulfate, albuterol, leukotriene receptor antagonists; electrolytes, metals and minerals; antianxiety and antidepressant agents; antimicrobial and antiviral agents; antihistamines; immune-suppression agents; cholesterol-lowering agents; cardiac and high-blood pressure agents and mixtures thereof.

This larger and quicker release of this invention will allow medication to be delivered to a patient much quicker and in more exact prescribed quantities. This burst electrode system may find use in a transdermal pad medication system, wherein a patient wearing said transdermal pad containing the burst electrode system contained therein receives doses of medicine through no exertion on the patients behalf.

### EXAMPLES

The examples herein are illustrations of various embodiments of this invention and are not intended to limit it in any way.

Pyrrole and N-methyl pyrrole (Aldrich) were passed over neutral alumina until colorless before use. ATP disodium salt (Sigma Chemical), dopamine, epinephrine (Acros), metaproternol (Sigma) and Na PSS (ALCO) were used without further purification. Electropolymerization and redox switching studies were carried out in a single compartment cell using an EG&G Model 273 potentiostat. UV-Vis absorbance studies were carried out using a Cary 5 E UV-VIS-NIR spectrophotometer.

Anion loaded films were prepared by the direct electropolymerization of pyrrole and N-methyl pyrrole in the anion containing electrolyte, providing materials which could release the anions upon reduction. PPy/PSS electrodes were prepared and loaded by reducing the films in aqueous solutions of the cationic biomolecules. The release properties of the loaded electrodes were probed in phosphate buffer (pH=7.4) in order to provide a biological medium. We have found that we can prepare electrodes that supply controlled amounts of the active molecule by controlling the amount of material on the electrode surface. In addition, the high reactivity of polypyrrole serves to yield materials with burst release properties where significantly more drug can be released rapidly from the system than expected from an electrochemically well-behaved Faradaic material.

I. Polypyrrole

Cationic Drug Systems

For cation loading experiments, PPy/PSS films were prepared on stainless steel (3 cm²) by constant current polymerization at 0.25 mA/cm² using 0.04 M pyrrole and 0.1 M PSS in distilled water for ca. 1 hour. The electrolyte/monomer solutions were purged with argon prior to use and experiments carried out under an argon blanket. While argon was utilized, one of ordinary skill in the art would recognize that other gases (such as nitrogen) may also be utilized to purge the electrolyte/monomer solutions. Further, purging may not be necessary in industrial type applications as polypyrrole coated textiles are made without purging (such as by Milliken of Greenville/Spartanburg, S.C.). The electrodes were polished, wiped with a tissue, and washed distilled water prior to each experiment. Film thickness was controlled by the amount of charge consumed for the electropolymerization and measured via profilometry. The films were washed thoroughly with water to remove excess monomer and electrolyte, and subsequently transferred to an aqueous solution containing only protonated drug molecules.

Drug loaded electrodes were produced by constant potential reduction at −0.5 V vs Ag/AgCl in a 0.1 M aqueous solution of the above hydrochloride salts allowing the current to decay to background. After loading, the polymer electrodes were washed with deionized water and placed in 7 mL phosphate buffer (20 mM, pH 7.4). Electrochemically stimulated release experiments were carried out using constant current, constant potential, or pulsatile (both current and potential) methods.

As described earlier, the use of electrostatically-bound doped anions, most often polyelectrolytes, yield electroactive polymer films with cation-dominant transport characteristics. Using stainless steel electrodes in aqueous NaPSS electrolyte, conditions were developed for the reproducible synthesis, loading, and release of bioactive cations from PPy/PSS films. After PPy/PSS film preparation, the polymer-modified electrodes were removed from the electrolyte, washed with water, and their redox properties studied by cyclic voltammetry (CV). Comparing NaCl and dopamine hydrochloride electrolytes, it can be seen that both sets of cyclic voltamograms exhibit broad, anodic peaks at about +0.2 V with corresponding cathodic processing peaking at ca. −0.4 V. As expected for well-behaved, surface supported, electroactive films, both the cathodic and anodic current responses are linearly dependent on scan rate. The nearly identical CV response in these two cases demonstrates the high electroactivity of PPy/PSS in the dopamine-based electrolyte. In addition, over this potential range, no significant current response is evident due to dopamine oxidation. Dopamine was found to oxidize at bare metal electrodes at +0.6 V vs. Ag/AgCl under a nitrogen atmosphere. Not to be bound by theory, it is speculated that either it does not react or its oxidation is very slow at the PPy/PSS modified electrode surface. As these electrodes will be used for dopamine release, the stability of the dopamine is important. Similar CV experiments were carried out for a prior-loaded PPy/PSS-dop⁺ film in which the dopamine was pre-loaded by application of a constant potential of −0.5 V in 0.1 M dopamine.

To test the stability of the PPy/PSS-dop⁺ to spontaneous ion exchange, films were placed in 20 mM phosphate buffer (pH=7.4) for 96 hours without stirring. A UV/V is spectrum of the electrolyte after this exposure shows that the polymer is stable to spontaneous release as no peak absorbance for dopamine is observed. A second film, prepared under identical conditions, was rinsed and cycled between +0.2 V and +1.2 V at 25 mV/s in phosphate buffer for ten cycles. The large absorbance at λmax=280 nm, indicates that the dopamine was rapidly electrochemically expelled from the film. These results suggest that ca. 95% of the dopamine that was initially loaded could be released during this experiment.

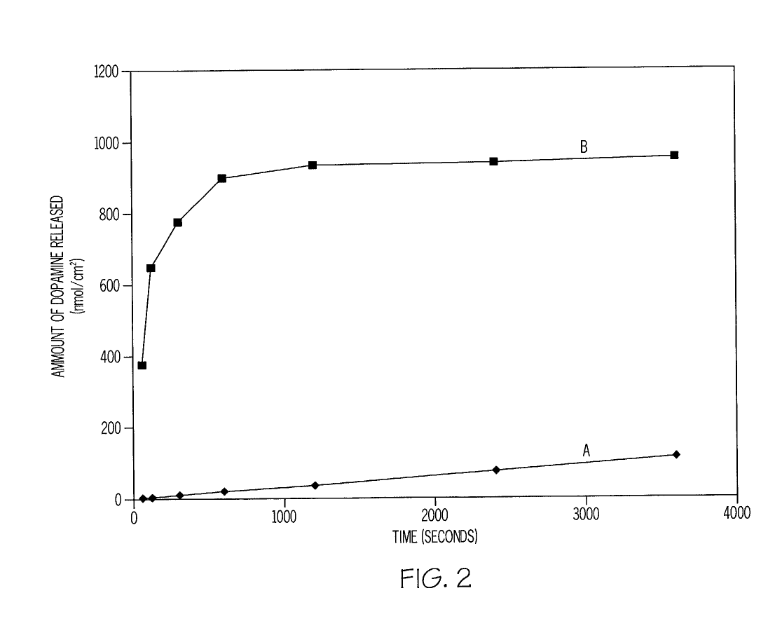

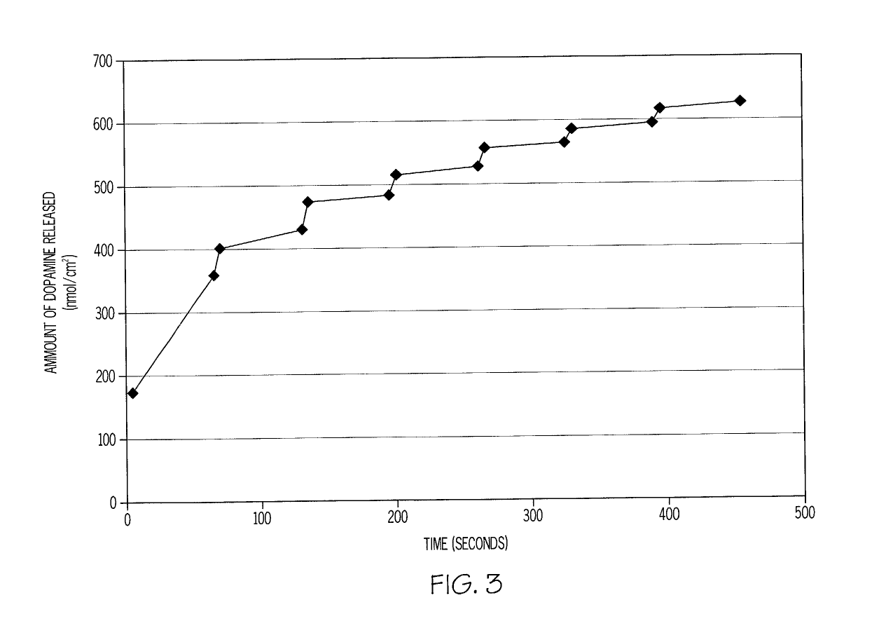

We find that PPy/PSS-dop⁺ electrolytes can be used to release dopamine when they are subjected to both constant current or constant potential electrochemical stimuli in phosphate buffer. As shown in FIG. 2, when a constant current of 3.0 μA/cm² was applied, essentially all of the dopamine was released within 300-600 seconds. Using 2.9 micron thick films, the dopamine content released is approximately 900 nmol/cm². Also shown in FIG. 2 is the expected dependence of the release if the system behaved Faradaically. It can be seen that the actual rate of dopamine released was significantly faster than that expected Faradaically, and that a large amount of dopamine released with a very small net amount of charge. We have classified this behavior as “burst” release and detailed examples are shown below. Experiments showed that different constant current or applied potential values have a very minimal effect on the total amount of dopamine released and, in fact, the release rate during application of the electrochemical stimulus is relatively constant. In order to determine the potential applicability of the PP/PSS-dop⁺ electrodes for pulsatile dopamine release experiments, the dopamine electrodes were placed in a phosphate buffer and a constant current pulse of 0.33 μA/cm² was applied for 5 seconds, followed by an open circuit period of 60 seconds. As can be seen from FIG. 3, when the initial current pulse was applied, a significant amount of dopamine was immediately released due to the burst effect. After two pulses, approximately two-thirds of the releasable dopamine had been expelled. As this current pulse corresponds to 1.65 μC/cm², only a small amount corresponding to 0.017 nmol/cm² of dopamine would have been expected to be released from a Faradaically well-behaved system. Since we observed that about 400 nmol/cm² of dop⁺ released after two pulses, the system is not behaving Faradaically. It was found that varying the current and potential of these pulses had no effect in controlling the amount of active molecules released, and it is likely that some chemical effect occurred first. Not to be bound by theory, we attribute this to the extreme oxidative instability of PPy and, even with careful handling of the polymer, it becomes partially oxidized. While the partially oxidized material does not spontaneously release, the electrochemical stimulus opens the membrane (in essence “bursting the bubble”) and the dopamine leaves. At the same time, at open circuit there is a negligible amount of dopamine released after the second pulse. After several pulses, the system becomes better behaved with incremental amounts of dopamine released with each pulse. While fully loaded PPy/PSS-dop⁺ membranes are inappropriate materials for pulsatile release applications via multiple potential or current pulses, the burst release behavior may prove useful. Electrodes displaying this burst release characteristic can be made to rapidly and efficiently deliver a prior-determined amount of drug with a very high electrical efficiency. This may be especially beneficial in situations, which are limited in the amount of charge that can be delivered to a system.

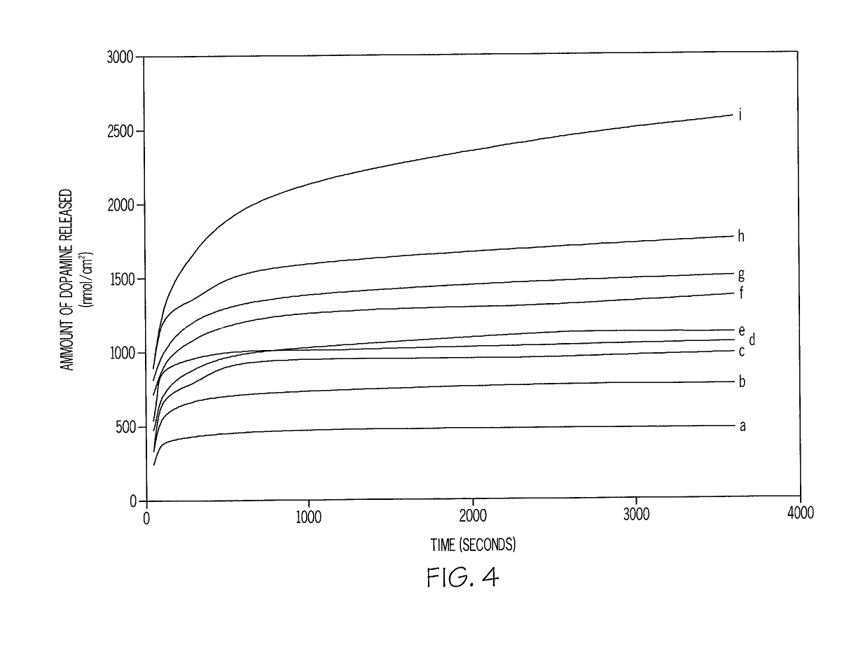

To demonstrate this, we subsequently developed a method for controlled release by varying the film thickness of the originally-deposited PPy/PSS and thus the molar content of loaded dopamine per unit are of electrode. PPy/PSS-dop⁺ films were prepared with varied thicknesses, ranging from 1.4 microns to 19.1 microns, in order to release different amounts of dopamine to solution. Loaded films prepared in this manner were placed in phosphate buffer and time dependent release was monitored at a current of 3.3 μA/cm². As shown in FIG. 4, facile control of dopamine release is easily obtained, and we can vary the total amount of dopamine released form 0.5-2.5 μmol/cm² of electrode area. These experiments suggest that it may be quite easy to control the amount of dopamine released in a practical system by varying the film thickness of the electroactive polymer membrane.

Epinephrine

Using the same film preparation and loading conditions as developed for PPy/PSS-dop⁺, epinephrine loaded PPy/PSS-epi+ films were subsequently prepared. Epinephrine was found to exhibit the same loading and release behavior as dopamine. At a constant current of 3.3 μA/cm², approximately 350 nmol/cm² of epinephrine within a few minutes without subsequent release thereafter. Again, the release rate is significantly faster than that expected from a Faradaically behaved system and the material behaves as a burst release membrane.

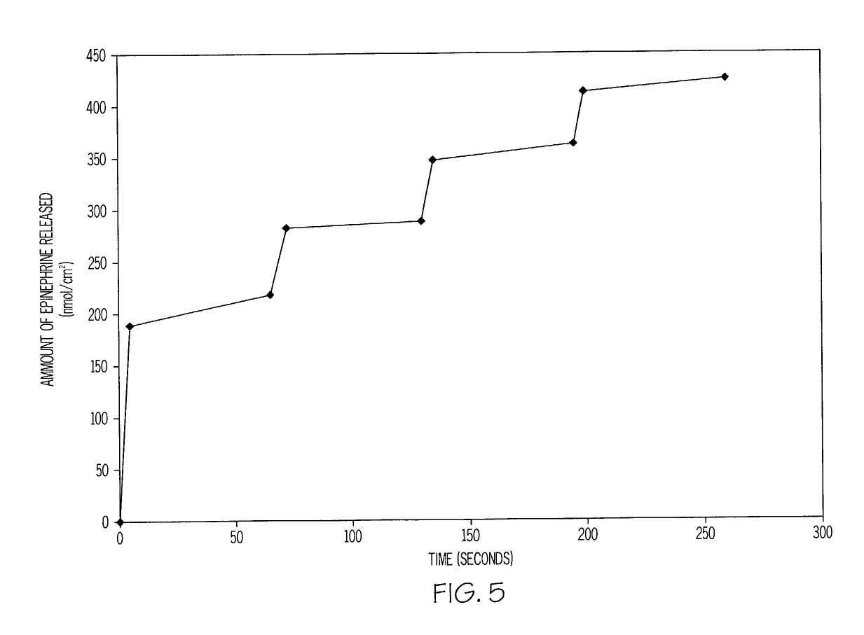

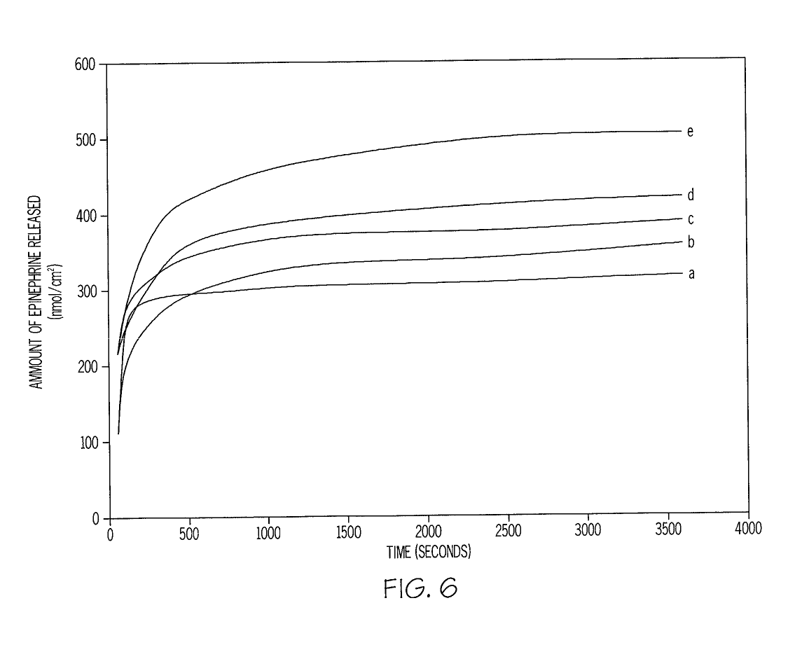

As with dopamine, pulsatile release of epinephrine from PPy/PSS led to the emission of a large amount of active molecule from the films during the first pulse. In this instance, approximately 50% (200 nmol/cm²) of the total epinephrine loaded is released in the first burst as shown in FIG. 5 where only 0.17 nmol/cm² would be expected from Faradaic release. While multiple plateaus can be reached during the pulsing, only a minimal number of pulses are possible due to the rapid release of epinephrine. Again, by analogy with the controlled release of dopamine, we have used different film thicknesses of PPy/PSS as a means to control the amount of epinephrine released, as shown in FIG. 6. In this study, films ranging in thickness form 2.2-7.2 μm were found to release between 300 and 500 nmol/cm² of the drug.

Metaproterenol

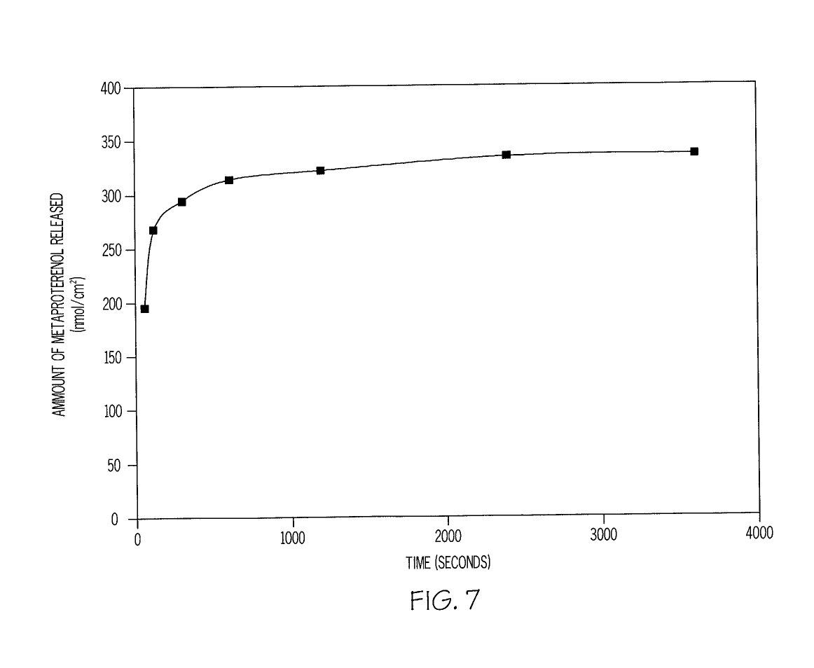

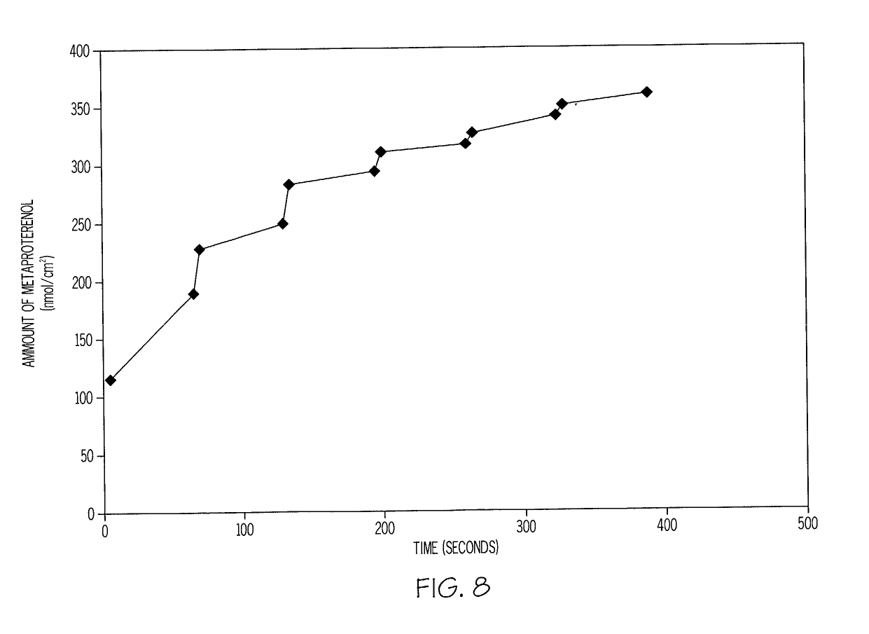

Metaproterenol was used as an active molecule to be loaded and released. As with both the other catecholamine neurotransmitters studied, metaproterenol could be loaded and released from PPy/PSS in a similar manner. As seen in FIG. 7, approximately 320 nmol/cm² of metaproterenol releases from a 2.9 μm thick film within a few minutes upon supplying a current of 3.3 μA/cm². In this instance, the amount of the metaproterenol released is only a fraction of that seen for dopamine and epinephrine. This may be attributed to the larger molar volume of the metaproterenol. Pulsatile release of metaproterenol was similar to that of epinephrine and dopamine (FIG. 8) in that burst release of the drug was observed upon the initial electrochemical stimulus, followed by smaller controlled amounts with subsequent pulses.

Anionic Drug Systems

PPy/ATP films were synthesized at constant potential (0.8 V vs. Ag/AgCl) from an aqueous solution of 0.1 M pyrrole and 20 mM ATP (solution pH 3.2). The solution was purged with argon prior to use and all experiments were carried out at room temperature under an argon atmosphere unless otherwise specified. As noted above, while argon was utilized, one of ordinary skill in the art would recognize that other gases such as nitrogen may also be utilized to purge the electrolyte/monomer solutions. Further, purging may not be necessary in industrial type applications as polypyrrole coated textiles are made without purging such as by Milliken (Greenville/Spartanburg, S.C.). The working electrodes were either stainless steel or platinum foil, while Ag/AgCl was used as a reference electrode. A polymerization time of ca. 20 minutes was used to obtain films with a charge density during deposition of 625 mC/cm². Film thickness was measured using a Sloan Dektak II profilometer. Release experiments were carried out in a phosphate buffer (pH 7.4), NaClO₄ (pH 5.6) or NaCl (pH 5.6) at constant potential.

Polypyrrole-Adenosine triphosphate (PPy/ATP)—In order to determine the possibility of binding and release of a multi-charged, large anion, ATP has been used as the dopant for polypyrrole, avoiding the use of other electrolytes during electropolymerization. ATP is incorporated efficiently during the polymerization/deposition process and the films prepared show a uniform electrode coverage and composition. The use of a conductive polymer as an ion release agent will be limited if spontaneous release process dominate in electrolytic solutions. Exposure of pre-conditioned PPy/ATP films to either NaCl or NaClO4 (pH 5.6) solutions led to no visible spontaneous release after 17 hours as monitored by solution absorbance of the medium at 260 nm. At this pH, a gradual, yet minimal, release is observed for extended time periods. For example, after two weeks in the electrolyte between 1-5% of the ATP is spontaneously released. Raising the pH of the medium to 7.4 by using a phosphate buffer led to faster spontaneous release characteristics. Approximately 200 nmol/cm2 was released after buffer exposure for 17 hours. As this corresponds to approximately 66% of the total ATP initially incorporated into the film, these spontaneous release characteristics will require repression in useful devices. It is evident that the higher pH favors the dissociation of the weakly acidic ATP and thus, PPy/ATP can be used for electrochemically stimulated release at low pH with extended exposure, or at a higher pH with little long-term exposure to the electrolytic medium.

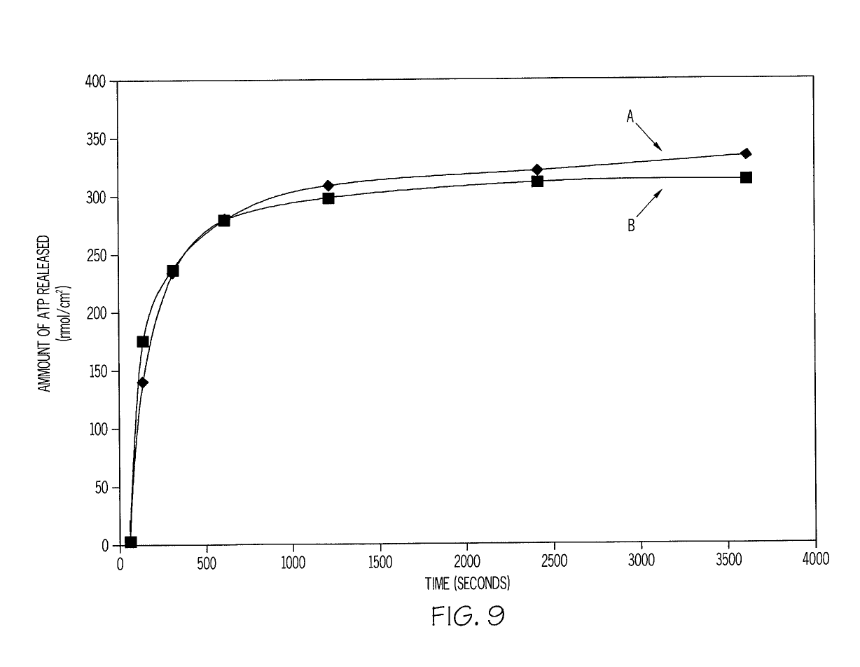

PPy/ATP films were subjected to constant potential release immediately after synthesis and washing by applying −0.5 V to the film for 1 hour. As shown in FIG. 9, there is an immediate release of the ATP into the electrolyte, leveling off at ca. 310 nmol/cm² after 20 minutes. This release content is highly reproducible with a final release amount varying by ±5% for different samples. While it is not possible to store these electrodes in the buffer medium due to spontaneous ATP exchange, we find the release characteristics of the films to be relatively stable to storage in an inert atmosphere. Films prepared using the same conditions as above were stored in argon for seventeen hours and subjected to the same release conditions, and yield the results shown in FIG. 9. It can be seen that the overall release characteristics are quite similar and, within experimental error, argon storage has no effect on the films release characteristics.

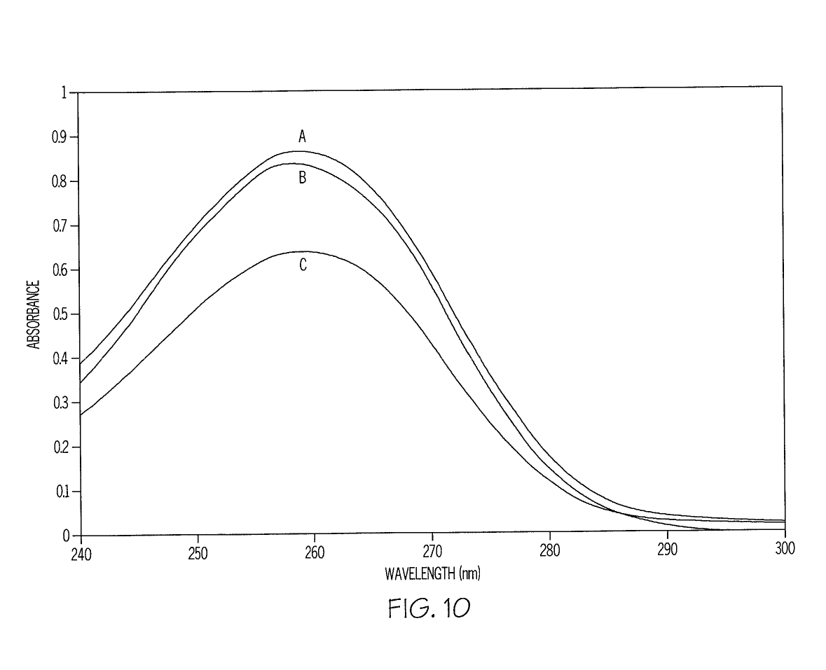

ATP release can also be effected by potential cycling between the doped and undoped states of the polymer, serving to drive the ATP from the film during the low potential portion of the cycle. FIG. 10 shows the UV/Vis spectrum of a 6×10⁻⁵ M ATP standard solution. It can be seen that a similar concentration of ATP was released when a film was cycled ten times between −1.0 V and 0.0 V at 20 mV/s in phosphate buffer as shown in FIG. 10. It is interesting to note that this electrically-driven process requires ca. 20 minutes for this release while approximately the same amount of ATP requires 17 hours to be spontaneously released. This suggests that, using a specific electrolyte, a limited fraction of the ATP is accessible and releasable. When a PPy/ATP electrode is cycled in 0.1 M aqueous NaClO₄, it displays similar release behavior as shown in FIG. 10, though slightly more ATP can be released. Although this electrode is relatively stable to spontaneous ion exchange processes, appreciable amounts of ATP could be released with potential cycling over a shorter time period.

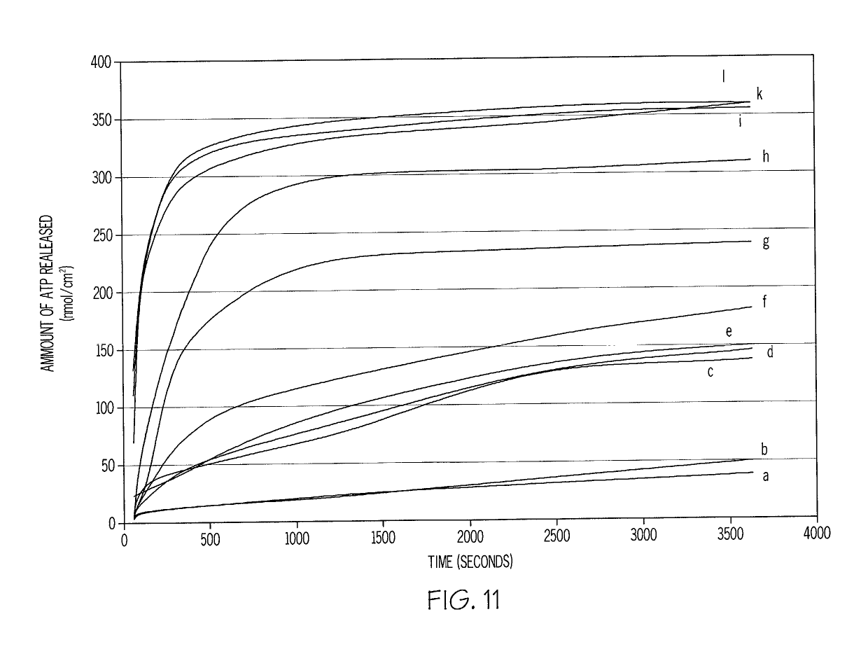

Electrochemically-controlled drug release systems will prove useful if the amount and rate of the active molecule to be released can be controlled using standard electrochemical parameters (e.g. current, potential, etc.). In order to determine the electrode potential dependence of ATP release, PPy/ATP films, prepared under the same conditions as above, were subjected to constant potential release at applied potentials ranging from −0.1 to −0.8 V in 0.1 M NaCl as shown in FIG. 11. It can be seen that only slow release occurs when the potential is held anodic of −0.2 V and the ATP tends to remain entrapped in the film. As the potential is shifted cathodically, the PPy begins to reduce and the ATP is released into the electrolyte between −0.2 and −0.3 V where the PPy/ATP turns into an effective ATP release electrode. Both the rate of release and the total amount of ATP released can be increased by using more cathodic release potentials with full release attained between −0.6 V to −0.8 V.

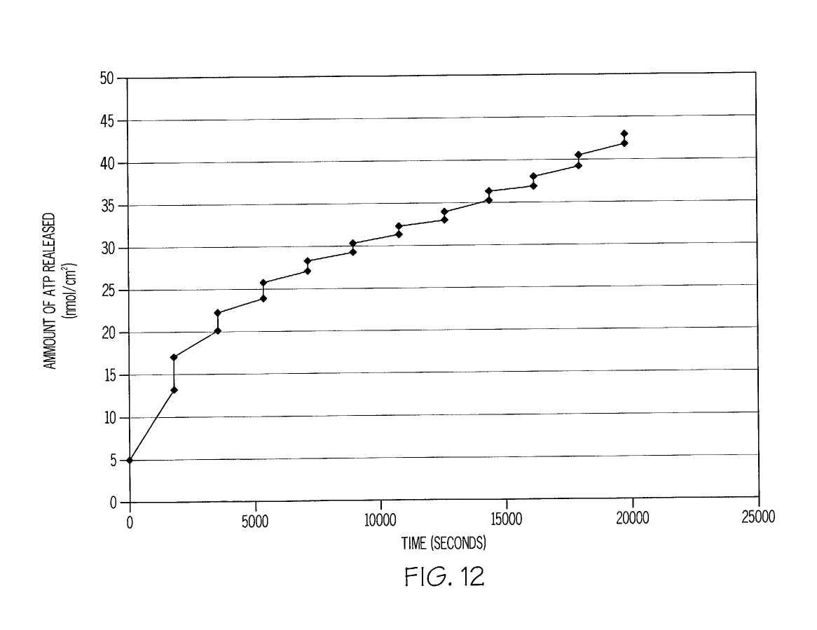

An important consideration in controlled drug release is whether the drug can be delivered at a slow rate, or in small increments over a long period of time. Application of an electrical stimulus to an electroactive membrane is especially well-suited for pulsatile release as the relatively rapid electrochemical impulse (current pulse, potential step) can subsequently be followed by diffusion of the drug from the membrane into the medium of interest. It is important in these situations to develop conditions in which a controlled amount of material is delivered within a certain time frame which can be followed by an acquiescent period where no drug is released. To examine these properties in PPy/ATP, release experiments were carried out by applying −0.25 V for 5 seconds and subsequently monitoring the ATP release with the electrode held at +0.5 V for 30 minutes. This is shown in FIG. 12 for a series of 12 repeated potential steps. During the initial 3 or 4 steps a relatively large amount of the total ATP is released, but subsequently a relatively constant and small amount is released per step. The poising of the electrode at +0.5 V between steps holds the polymer in its oxidized form and thus, no further electrochemically-driven ATP release should occur. The continued release of ATP for up to 30 minutes suggests that simple diffusion of ATP from the film is quite slow.

Poly(N-Methyl Pyrrole)/Poly(Styrene Sulfonate) (PNMPy/PSS)

In the experiments carried out on PPy described above, there was no spontaneous release observed for electrodes placed into a buffer solution for prolonged periods. After this storage, the catecholamine drug could not be electrochemically released from the PPy suggesting a possible reaction between the polymer and the incorporated drug. While the nature of this reaction is not presently known it is possible that the N—H of the pyrrole can be hydrogen bonded, with the loaded drug and, with time, an irreversible binding could occur. For this reason, we chose to investigate PNMPy as an electroactive polymer in which to load catecholamine drugs. As the nitrogen is methyl-substituted, hydrogen bonding interactions between the polymer and the drug will be limited. In addition, neutral PNMPy is significantly more stable to oxidation than PPy thus avoiding side reactions and allowing the burst release mechanism to be studied in detail.

PNMPy/PSS-dop⁺ films were prepared using the same conditions as developed for PPy/PSS-dop⁺. While film preparation and loading characteristics were quite similar between the two systems, it was found that the PNMPy/PSS-dop⁺ spontaneously released most of the loaded dopamine within 24 hours upon exposure to aqueous electrolyte. As PNMPy has a significantly higher oxidation potential than PPy, it can be stored in both air or under inert atmosphere and continue to retain electrochemically-induced drug release properties. In order to probe the electroactivity of the PNMPy/PSS system, CV experiments were carried out in different electrolytes. Interestingly, the polymer was found to be electroinactive in NaCl electrolyte solutions, while exhibiting a similar electroactivity to PPy/PSS in phosphate buffer and dopamine-based electrolytes. As such, further experimentation on the system was carried out using a phosphate buffer as the electrolytic medium.

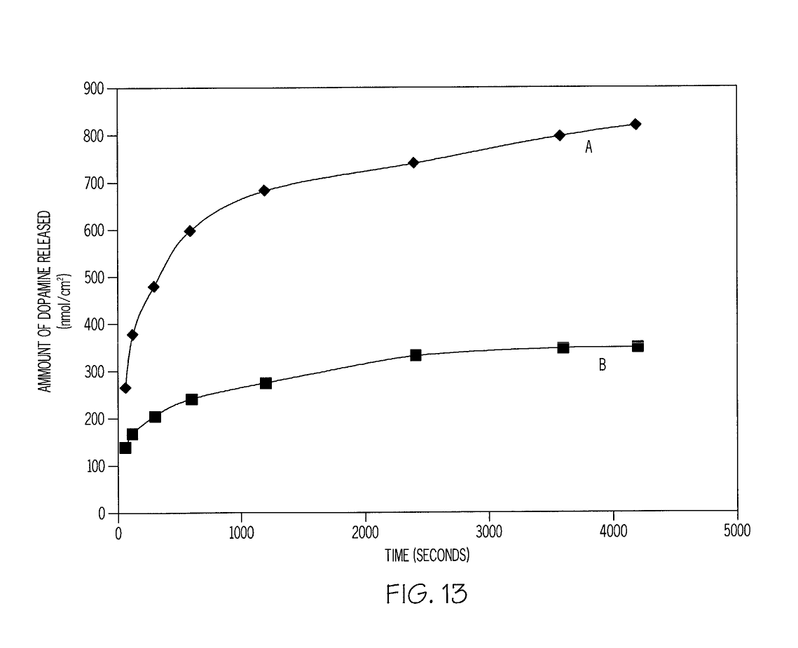

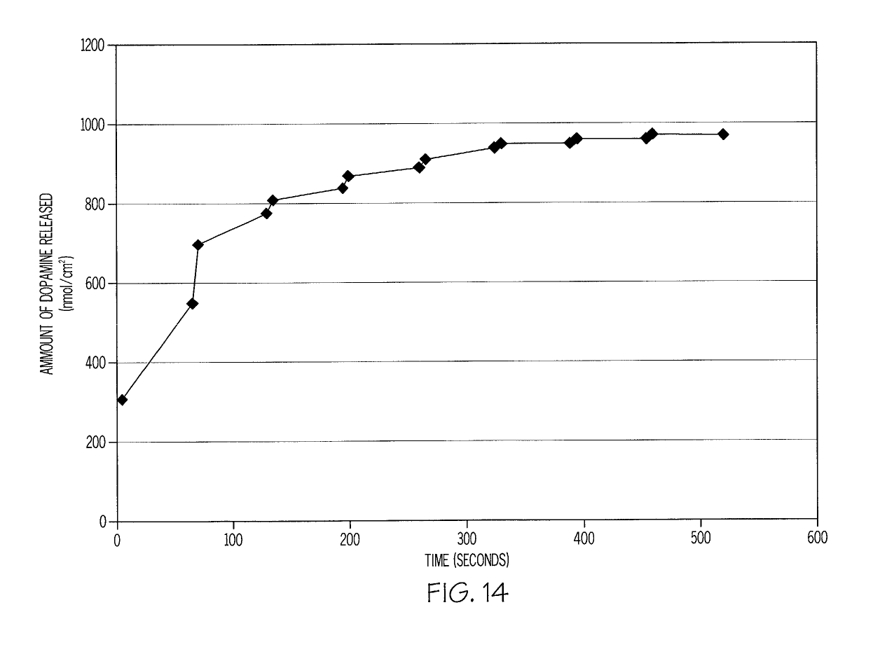

NMPy was electropolymerized from an aqueous solution of 0.04 M NMPy and 0.1 M PSS at a constant current of 2.7 mA/cm². The loading procedure was carried out at a constant potential of −0.6 V in a 0.1 M dopamine solution until the reductive current reached a plateau. Release experiments were subsequently carried out in 20 mM phosphate buffer (pH 7.4) at a constant current of 3.3 μA/cm². The release experiments, shown in FIG. 13, demonstrate that immediately after preparation the PNMPY/PSS-dop⁺ electrode can release a substantial amount of dopamine (ca. 800 nmol/cm²) in approximately ten minutes. A film prepared under identical conditions was stored under an argon atmosphere for ten days, and then subsequently exposed to the same release conditions. The results shown in FIG. 13 demonstrate that, while a lower amount of the dopamine was accessible for release, the film still retained its release characteristics. As mentioned above, this is in contrast to PPy/PSS-dop⁺ where this storage would have completely inhibited any release. Similar release properties were seen for PNMPy/PSS-dop⁺ films stored in air. Pulsatile release experiments were again attempted with this PNMPy/PSS-dop⁺ system but, as seen with the PPy system, an initial large burst of dopamine is released upon application of any electrical stimulus. This is illustrated by FIG. 14 where the first two pulses release approximately 700 nmol/cm² of the dopamine with ca. 1000 nmol/cm² being released after ten pulses.

Comparing and contrasting the experiments outlined above, we find that PPy-based systems are well behaved for anionic drug release. The films can be stored in their oxidized and relatively stable state, making them easy to handle. Cationic drug release from reduced PPy films yields significant problems due to instability and subsequent reactions and binding of the drugs within the films. It is likely that, even with relatively careful handling under an inert atmosphere, the easily oxidized PPy is reacting and partially degrading. At the same time, this has led to a process we term “burst” release and this concept may be of future use as very small amounts of an electrochemical stimulus can be used to release large amounts of active molecules.

Turning to the PNMPy-based system, as there is no accessible proton for hydrogen bonding, the system spontaneously exchanges the drug molecules rapidly. The higher oxidation potential of the PNMPy polymer allows it to be stored in both its reduced and oxidized forms and subsequently used for electrochemically-induced cation release.

Thus, it is apparent that there has been provided, in accordance with the instant invention, a process that fully satisfies the objects and advantages set forth herein above. While the invention has been described with respect to various specific examples and embodiments thereof, it is understood that the invention is not limited thereto and many alternatives, modifications and variations will be apparent to those skilled in the art in light of the foregoing description. Accordingly, it is intended to embrace all such alternatives, modifications and variations as fall within the spirit and broad scope of the invention.

What is claimed is:

1. A burst electrode system comprising an electroactive polymer having thereon a biologically active ingredient releasable from said electroactive polymer, whereby said burst electrode system exhibits a non Faradaic biologically active ingredient release profile.

2. The system of claim 1 wherein said biologically active ingredient is an anion.

3. The system of claim 1 wherein said electroactive polymer contains a dopant.

4. The system of claim 3 wherein said biologically active ingredient is a cation and said dopant is a polyanion.

5. The electrode system of claim 1 wherein said non-Faradaic release profile is generated by application of a potential to said electrode system and said release is measured and compared with the amount of said applied potential generating said profile.

6. The electrode system of claim 1 and 2 wherein said electroactive polymer comprises a polypyrrole polymer.

7. The electrode system of claim 1 wherein the electroactive polymer comprises poly(N-methyl pyrrole).

8. The electrode system of claim 3 wherein the electroactive polymer comprises poly(N-methyl pyrrole).

9. The electrode system of claim 4 wherein the electroactive polymer comprises poly (N-methyl pyrrole).

10. A burst electrode system comprising an electroactive polymer having thereon a drug releasable from said electroactive polymer, whereby said burst electrode system exhibits a non Faradaic drug release profile.

11. The system of claim 10 wherein said polymer contains a dopant.

12. The system of claim 3 wherein the drug is a cation and the dopant is a polyanion.

13. The electrode system of claim 10 wherein said non-Faradaic release profile is generated by application of a potential to said electrode system and said release is measured and compared with the amount of said applied potential generating said profile.

14. The electrode system of claim 10 wherein said electroactive polymer comprises a polypyrrole polymer.

15. The electrode system of claims 10 wherein the electroactive polymer comprises poly(N-methyl pyrrole).

16. The electrode system of claim 10 wherein said drug comprises phenylpropanol, pseudoephedrine, hydrocortisone, metaproternol, polymyxin, chloropheniramine, and erythromycin.

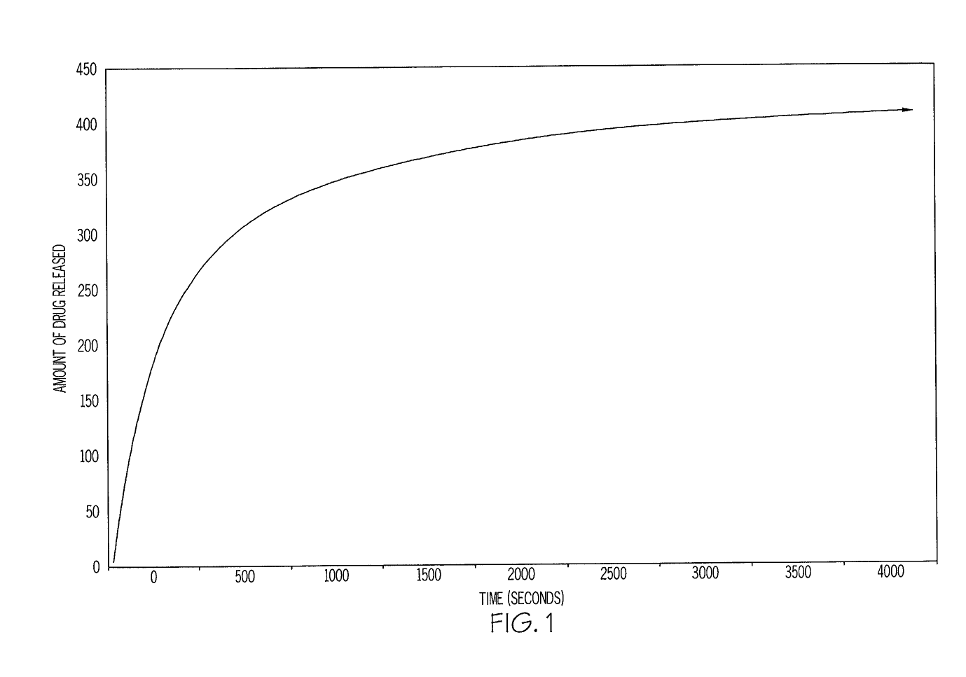

17. A burst electrode system comprising an electroactive polymer having thereon a drug releasable from said electroactive polymer, whereby said burst electrode system exhibits a drug release profile characterized by FIG. 1.

18. The burst electrode system of claim 17 wherein the drug is an anion.

19. The burst electrode system of claim 17 also comprising a dopant.

20. The burst electrode system of claim 19 wherein the dopant is a polyanion and the drug is a cation.

21. The electrode system of claim 17 wherein said profile is generated by a single burst of a current or potential which causes a single release of a larger quantity of the drug than would be expected from a Faradaic release.

22. The electrode system of claim 21 wherein said profile is characterized by the release of a disproportionately large amount of drug in proportion to voltage applied to said polymer.

23. The electrode system of claim 18 wherein said electroactive polymer comprises polypyrrole.

24. The electrode system of claim 20 wherein said electroactive polymer comprises polypyrrole/poly(styrene sulfonate).

25. The electrode system of claim 24 where a second polymer layer comprises an overlayer.

26. The electrode system of claim 25 where the overlayer is hydrophobic and crosslinked.

27. An article of manufacture comprising a burst electrode system having a non Faradaic drug release profile, which comprises an electroactive polymer containing a drug releasable from said electroactive polymer.

28. The article of manufacture of claim 27 further comprising a polyionic dopant.

29. The article of manufacture of claim 27 wherein said polymer comprises a polypyrrole or poly(n-methyl pyrrole).

30. The article of manufacture of claim 28 and 29 wherein said polyionic dopant is a polyanion and the drug is a cation.

31. The article of manufacture of claims 29 and 30 wherein said drug comprises catecholamines (dopamine, norepinephrine, or metaproterenol), phenylpropanol amine, chloropheniramine, salicylic acid, pseudoephedrine, dichlophenac, erythromycin, hydrocortisone, metaproternol, or polymyxin.

32. A method of treating a patient using a burst electrode system, which comprises a burst electrode system comprising an electroactive polymer, loading said electroactive polymer with a drug releasable from said electroactive polymer and contacting said patient with said electrode system in an effective contacting manner so as to trigger the release of said drug from said electroactive polymer, whereby said drug is made effectively available to said patient.

33. The method of claim 32 where the loading of said electroactive polymer is further accomplished with a polyanionic dopant.

34. The method of claim 33 wherein the drug is a cation.

35. The method of claim 32 wherein the drug is an anion.

36. A burst electrode system comprising an electroactive polymer having thereon a drug releasable from said electroactive polymer, whereby said burst electrode system exhibits a non Faradaic drug release profile.

37. The system of claim 36 also comprising a polyanionic dopant species incorporated into the electroactive polymer.

38. The method of claim 36 where the reaction wherein the drug is released during a reduction reaction.

39. The method of claim 37 wherein the reaction wherein the drug is released is an oxidation reaction.

40. The method of claim 38 wherein the drug released is an anionic drug.

41. The method of claim 39 wherein the drug released is a cationic drug.

42. The method of claim 41 wherein the drug is selected from the group consisting of salicylate, glutamate and ATP.

43. The method of claim 40 or 41 wherein the drug is a selected from the group comprising catecholamines (dopamine, norepinephrine, or metaproterenol), phenylpropanol amine, chloropheniramine, salicylic acid, pseudoephedrine, dichlophenac, erythromycin, hydrocortisone, metaproternol, or polymyxin.

44. A process whereby the release of a various biologically active molecules are electrochemically stimulated from electroactive conducting polymers.

45. The process of claim 44 wherein the electroactive conducting polymers are selected from the group consisting of polypyrrole, poly(N-methyl pyrrole), substituted polypyrrole, polythiophene, polydioxythiophene and polyaniline.

46. The process of claim 44 wherein the films are loaded with either an anionic drug species or a polyanionic species as a dopant with a cationic species as a drug.

47. The process of claim 46 wherein the biologically active molecules are incorporated into the electroactive conducting polymers by using ions as the charge compensating dopant during electropolymerization.

48. The process of claim 44 wherein the biologically active molecules are incorporated into the electroactive conducting polymers by redox switching of the polymer film in a bathing electrolyte containing the biologically active molecules.

49. The method of claim 47 wherein the biologically active molecules are drugs.

50. The method of claim 48 wherein the biologically active molecules are drugs.

51. The process of claims 49 wherein the drugs are selected from the group comprising dopamine, norepinephrine, metaproterenol, phenylpropanol amine, chloropheniramine, salicylic acid, pseudoephedrine, dichlophenac, erythromycin, hydrocortisone, metaproternol, or polymyxin.

52. A method for preparing a burst electrode system wherein said process comprises electropolymerizing pyrrole by constant current polymerization in polymerizable pyrrole and polystyrene sulfonate composition to form a polymer, loading a releasable drug on said polymer by constant potential reduction of said drug with said polymer in a composition to form an initial electrode system and thereafter removing said initial electrode system from said solution to allow equilibration of potential of said polymer outside said solution.

53. A method for preparing a burst electrode system wherein said process comprises electropolymerizing pyrrole by constant current polymerization in polymerizable pyrrole and polystyrene sulfonate composition to form a polymer, loading a releasable drug on said polymer by constant potential oxidation of said drug with said polymer in a composition to form an initial electrode system and thereafter removing said initial electrode system from said solution to allow equilibration of potential of said polymer outside said solution.
